(19) United States
(12) Patent Application Publication
(10) Pub. No.: US 2022/0009895 A1
(43) Pub. Date: Jan. 13, 2022

(54) N-NITROSACCHARINS

(71) Applicant: ETH ZURICH, Zürich (CH)

(72) Inventors: Dmitry KATAYEV, Zürich (CH); Kun ZHANG, Zürich (CH); Roxan CALVO, Zürich (CH)

(73) Assignee: ETH ZURICH, Zürich (CH)

(21) Appl. No.: 17/288,795

(22) PCT Filed: Oct. 24, 2019
(86) PCT No.: PCT/EP2019/079040

§ 371 Date: Apr. 26, 2021

(30) Foreign Application Priority Data
Oct. 26, 2018 (EP) 18202996.7

(51) Int. Cl. C07D 275/06

(52) U.S. Cl. CPC C07D 275/06

(57) ABSTRACT

N-nitrosaccharin of the general formula (I),

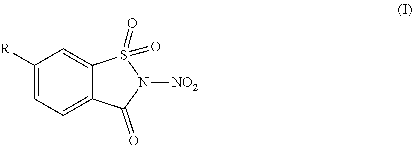

wherein R is either a hydrogen (H) or a nitro group (NO₂), its preparation and its use as nitrating agent.

The invention relates to new compounds and their use as nitrating agents.

Today, aromatic nitro compounds are almost exclusively synthesized by electrophilic nitration of arenes using a large excess of the nitric acid or mixed strong-acid systems, such as H₂SO₄/HNO₃. Such acidic reaction conditions (mixture of strong acids in combination with high reaction temperature up to 135° C.) represent a limitation in terms of tolerance towards acid-sensitive functional groups and result in the selectivity problem leading to the formation of complex mixtures of regioisomers and over-nitrated side products.

CN 104 945 304 B discloses a process for trifluoromethylthio arene production using trifluoromethylthiosaccharin.

Cochet et al. (Cochet et al., Synlett, vol. 2011, no. 13, pages 1920-1922) disclose N-formylsaccharin as a new formylating agent.

Ueda et al. (Ueda et al., 2013, Angewandte Chemie International Edition, vol. 52, no. 33, pages 8611-8615) use palladium to catalyze a reductive carbonylation of aryl halides with N-formylsaccharin as a CO source.

WO 2016/118450 A1 discloses the nitration process of aromatic compounds using concentrated nitric acid and an anhydride in absence of an organic solvent.

As one of the first derivatives 6-nitrosaccharin has been prepared by the method of Noyes in 1886. Since then, various other derivatives of nitrosaccharin have been synthesized that contained nitro groups in the 4- and 5-position (e.g. by G. H. Hamor in J. Am. Pharm. Ass. Vol. 49, No. 5, 1960).

With the term “N-nitrosaccharins” it is referred to saccharins containing a nitro group on the nitrogen. In the literature only two references have been noted with regard to primary attempts to synthesize N-nitrosaccharins.

The first reference of Runge and Treibs (Journal der praktischen Chemie, 1962) describes a procedure that uses N₂O₅, which is a solid compound (subliming at slightly above room temperature) and decomposes to NO₂ and O₂ at room temperature. The yield of the proposed product was only 14% after 6 days. As to the characteristics of the proposed product it was merely indicated that the substance decomposes at 170° C. and is soluble in dioxan but not in EtOH. In consideration that according to current state of knowledge the decomposition temperature of pure N-nitrosaccharin is actually much higher and due to the lack of further analytical data that would allow verification of the alleged synthesis of N-nitrosaccharin, it has to be assumed that another compound than N-nitrosaccharin was produced by Runge and Treibs.

The second reference is a scientific publication of Kozlova et al. that presents the synthesis of N-nitrosaccharin via reaction of saccharin ammonium salt with NO₂BF₄ (Kozlova, Lukyanov and Tartakovskii, Bulletin of the Academy of Sciences of the USSR, 1981). The nitration agent NO₂BF₄ is a very unstable compound and decomposes within seconds in air.

Despite multiple attempts to reproduce the procedure of Kozlova by the inventors of the present invention, the proposed synthesis was never successful since no product formation could be observed at all (described in experiment 5). Kozlova provided a melting point of 170° C. and reported an IR spectrum peak at 1610 cm⁻¹.

Therefore, it seems likely that neither of Runge/Treibs and Kozlova et al. has indeed been successful in their attempt to prepare N-nitrosaccharin, providing the poor evidence in order to conclude that a pure molecule of N-nitrosaccharin was obtained.

The object of the present invention is therefore to provide a reproducible and straightforward synthesis of N-nitrosaccharins. It is a further object of the present invention to use these new N-nitrosaccharins in a practical, safe, cheap and green process for the nitration of a compound comprising at least one substituted or unsubstituted aromatic or heteroaromatic ring.

This object is achieved by the provision of N-nitrosaccharins according to claim 1, by the method for their preparation according to claim 5 and by their use according to claim 12. Preferred embodiments of the invention are subject of the dependent claims.

The present invention is directed to N-nitrosaccharins of the general formula (I) below, wherein R is either a hydrogen (H) or a nitro group (NO₂).

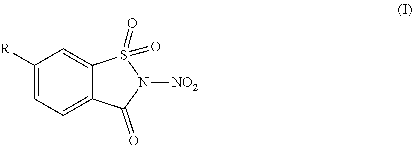

Specifically, the present invention pertains to unsubstituted N-nitrosaccharin of the formula (Ia) and 6-nitro-N-nitrosaccharin of the formula (Ib).

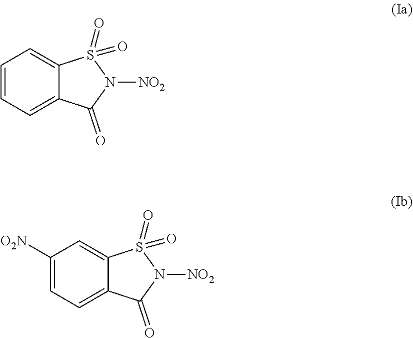 [SMILES: O=C1c2ccccc2S(=O)(=O)N1[N+](=O)[O-] ; O=C1c2ccc([N+](=O)[O-])cc2S(=O)(=O)N1[N+](=O)[O-]]

It has been surprisingly found that N-nitrosaccharins as defined above are superb electrophilic nitration reagents and are therefore of high value in the synthesis of compounds carrying one or more nitro groups.

In contrast to the postulated but not successful synthesis of N-nitrosaccharin via reaction of saccharin ammonium salt with the unstable and air-sensitive NO₂BF₄ suggested by Kozlova, the inventors of the present application found that N-nitrosaccharin and 6-nitro-N-nitrosaccharin can be prepared in a simple, one-step procedure under mild conditions with a high chemical yield of product. In addition, the reaction starts from commercially available saccharin or its derivates that are also readily available and low-cost commodities.

In a preferred embodiment of the invention N-nitrosaccharin of formula (I) is in crystalline form. Compound Ia comprises preferably monoclinic crystals and compound Ib comprises preferably orthorombic crystals. Crystalline compounds provide the benefit of high purity and their subsequent use. For the prepared N-nitrosaccharins of the present invention, no detectable decomposition was observed for storage for more than two months at ambient temperature. For N-nitrosaccharin white, colorless crystals were obtained. For 6-nitro-N-nitrosaccharin the crystals had a light-yellow (almost white) color. A decomposition point of 180-182° C. for N-nitrosaccharin of the formula (Ia) and 174-176° C. for 6-nitro-N-nitrosaccharin of the formula (Ib) was measured. Thus, N-nitrosaccharin are readily accessible, shelf stable for at least 2 months under air and easy-to-handle solid chemicals. Furthermore, compounds can be stored in freezer for at least 6 months without substantial decomposition.

In addition to the N-nitrosaccharins of the general formula (I) defined above the invention further relates to a method for their preparation. In accordance with the inventive method, N-saccharin of the general formula (II) is reacted with a mixture of nitric acid and acetic anhydride to obtain N-nitrosaccharin.

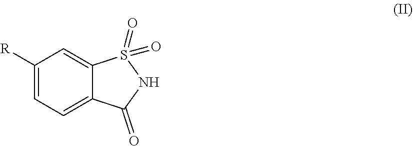

In the above general formula (II), R can either be a hydrogen or a nitro group. In case of R being a hydrogen, N-saccharin (II) is reacted with a mixture of acetic anhydride and nitric acid to produce N-nitrosaccharin (Ia). If R is a nitro group, the reaction of N-saccharin (II) with a mixture of acetic anhydride and nitric acid produces 6-nitro-N-nitrosaccharin (Ib).

In a preferred embodiment, concentrated nitric acid is used in the above-described preparation of N-nitrosaccharins. The use of concentrated nitric acid has shown to have a beneficial effect on the yield. The term concentrated nitric acid means a nitric acid solution of at least 15.8 M. Concentrated nitric acid is most preferred since the higher rates can be achieved.

In a preferred embodiment of the inventive process, N-saccharin is dissolved in organic anhydride before nitric acid is added. The organic anhydride is preferably selected from the group consisting of acetic anhydride, propionic anhydride, 2-methylpropionic anhydride, trimethylacetic anhydride, 2-ethylbytyric anhydride, butyric anhydride, fluoroacetic anhydride, trifluoroacetic anhydride and mixtures thereof.

Preferably, N-saccharin and the organic solvent, preferably acetic anhydride, are used in a molar ratio between within a range between 2:1 and 1:50, preferably between 1:1 and 1:20 and most preferably between 1:3 and 1:9.

In a further preferred embodiment of the process the molar ratios of N-saccharin to concentrated nitric acid is between 500:1 and 10:1, preferably between 200:1 and 20:1 and most preferably between 100:1 and 40:1.

In a further preferred embodiment of the process the solution of N-saccharin and aprotic solvent is cooled down below 15° C., preferably below 10° C. and most preferably below 5° C. during the addition of nitric acid. Since the addition of nitric acid on a large scale creates heat, it is preferable to regulate the temperature of the reaction mixture during the addition of nitric acid to maintain a constant and stable reaction environment.

In a further preferred embodiment of the process the reaction mixture is stirred for 1 to 24 hours, preferably for 2 to 10 hours and most preferably for 4 to 6 hours.

It is further preferred that a gas is bubbled through the reaction, preferably the gas is air and most preferably dry air, in order to remove excess of nitrogen oxides.

In a further preferred embodiment of the process, N-nitrosaccharin of the general formula (I) is obtained with a yield of at least 50%, preferably at least 75% and most preferably at least 90%, if N-saccharin of the general formula (II) is reacted with nitric acid.

Considering that the yield of comparable nitration reactions can be as low as 15% or even unsuccessful due to multiple nitrations or cross reactions with other reagents, the yields achieved by the inventive process are highly satisfying also in view of industrial application of the process.

In a further preferred embodiment of the process, the solvent is removed via filtration after completion of the reaction of N-saccharin nitric acid to obtain N-nitrosaccharin in crystalline form.

It has further been found that N-nitrosaccharin and 6-nitro-N-nitrosaccharin can be used as nitrating agent of an at least one substituted or unsubstituted aromatic or heteroaromatic ring in an electrophilic substitution. N-nitrosaccharins are bench-stable, can be prepared on a big scale in one chemical step within a few hours from cheap, commercially available chemicals.

The present invention therefore further relates to the use of N-nitrosaccharin of the general formula (I),

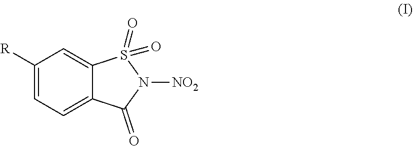

with R being either hydrogen or a nitro group, as nitrating agent of a compound A in an electrophilic substitution. Compound A thereby comprises at least one substituted or unsubstituted aromatic or heteroaromatic ring and said ring has preferably at least one heteroatom selected from the group consisting of oxygen, sulfur, phosphor, nitrogen and selenium.

Due to the presence of the additional electron-withdrawing nitro group, 6-nitro-N-nitrosaccharin reacts considerably more powerful and accelerates the nitration reaction compared to N-nitrosaccharin. 6-Nitro-N-nitrosaccharin is therefore particularly useful for use in the nitration of less activated starting materials.

In a preferred embodiment of the inventive use, the electrophilic substitution is an ipso-substitution. An ipso-substitution is a special case of an electrophilic aromatic substitution where the leaving group is not hydrogen. Thus, in this case, compound (A) comprises at least aromatic or heteroaromatic ring, said aromatic or heteroaromatic ring comprising a leaving group and may comprises further residues or not. Typically, said leaving group allows to form a stable carbocation intermediate.

Within the context of the present invention, the term heteroaromatic ring stands for a ring comprising at least one heteroatom selected from the group consisting of oxygen, sulfur, phosphor, selenium and nitrogen. Compound (A) comprises the at least one substituted or unsubstituted ring as structural part of a bigger complex molecule or it only consists of said at least one unsubstituted or substituted aromatic ring. Thus, the expression “compound (A)” encompasses arenes and heteroarenes as well as compounds comprising one or more aromatic or heteroaromatic rings in their chemical structure, such as for example estrone, estradiol and estriol. If more than one aromatic or heteroaromatic ring is present, said rings may be fused together or being connected through a bond such as an alkylene group or the like with each other. In other words, compound (A) can be a small, medium or large organic compound comprising or consisting of a substituted or unsubstituted aromatic or heteroaromatic ring.

In particular in view of ipso-substitutions it is preferred, that the leaving group Y is selected from the group consisting of halogen atoms (I, Br, Cl, F), SO₃H, Si(CH₃)₃, tosyl, mesyl, nosyl, brosyl, tresyl, dansyl, trifyl, hydroxides, alkoxides, amides, acetyl substituents and tert-alkyl substituents.

In the following section, the synthesis and characterization of unsubstituted N-nitrosaccharin and 6-nitro-N-nitrosaccharin will be described.

N-Nitrosaccharin can be prepared in a quantitative, one-step procedure from commercially available saccharin or its derivatives, that are readily available and low-cost commodity.

For the preparation of non-substituted N-nitrosaccharin, saccharin as starting material was added to a mixture of concentrated nitric acid and acetic anhydride at preferably below 10° C. After 4-5 hours, depending on the scale of the reaction, the desired reagent is precipitated as a white crystalline compound.

No detectable decomposition of this molecule was observed after storage for more than two months at ambient temperature under air in the presence of light. Thus, N-nitrosaccharins are readily accessible, shelf stable and easy-to-handle solid chemicals.

### EXAMPLE 1

The procedure for synthesis of N-nitrosaccharins:

In a 250 mL three necked round bottom flask equipped with dropping funnel, air outlet and stirring bar was placed N-saccharin (10.0 g, 54.64 mmol) in acetic anhydride (25.7 mL, 0.27 mol). The solution was cooled to 0-5° C. with ice-bath and concentrated nitric acid (25.1 mL, 0.61 mol) was added dropwise to the solution during 30 minutes, while dry air being bubbled through the solution rapidly in order to remove excess of nitrogen oxides. N-saccharin was completely dissolved once all nitric acid was added. The cooling bath was removed, and the reaction mixture was stirred at room temperate during at least 4 hours with continuous bubbling of air through the liquid. The precipitate which had formed during the reaction was collected on a sintered glass filter and dried under high vacuum until dryness (11.8 g, 95% yield). The material can be recrystallized from hot chloroform or acetonitrile and is a white crystalline compound. No decomposition of N-nitrosaccharin found after 24 hours at room temperature in CH₂Cl₂, CHCl₃, acetone, HFIP, THF, MeCN, benzene. Full or partial decomposition of N-nitrosaccharin was found in DMF, DMSO and MeOH.

M.p./Decomposition temperature 180-182° C. (mass loss −50%, determined by termogravimetric analysis);

¹H-NMR (300 MHz, CD₃CN): δ=8.05 (dt, J=7.4, 1.5 Hz, 1H), 8.14 (dt, J=6.1, 1.4 Hz, 1H), 8.16-8.23 (m, 2H);

¹³C-NMR (75 MHz): δ=121.7, 123.1, 126.5, 134.4, 135.9, 137.6, 151.7;

IR (ATR, neat): 3097, 1781, 1717, 1601, 1463, 1292, 1176, 1068, 1007, 891, 758, 662, 582, 500;

HRMS (El) m/z calculated for C₇H₄N₂O₅S: [M+] 227.9836, found 227.9842.

Analytically calculated for C₇H₄N₂O₅S: C 36.85, H 1.77, N 12.28 found: C 36.88, H 1.87, N 12.41.

Colorless crystals of compound Ia were obtained by slow evaporation from a saturated solution in chloroform/acetonitrile 1:1.

Crystal data and structure refinement of compound IA:

Empirical formula C₇H₄N₂O₅S

Formula weight 228.18

Temperature/K 100.0

Crystal system Monoclinic

Space group IT number 15

Space group name C 1 2/c 1

a/Å 12.1513(6)

b/Å 10.0288(5)

c/Å 14.5625(8)

α/° 90.0

β/° 102.979(2)

γ/° 90.0

Volume/Å³ 1729.29(15)

Z 8

ρ_{calc}g/cm³ 1.753

μ/mm⁻¹ 0.378

F(000) 928.0

Crystal size/mm³ 0.076×0.199×0.202

Radiation Monα (λ=0.71073)

2θ range for data 2.662 to 28.282

collection/°

Index ranges −16≤h≤16, −13≤k≤13, −19≤1≤19

Reflections collected 35975

Independent reflections 2154

Data/restraints/parameters 2154/0/136

Goodness-of-fit on F² 1.101

Final R indexes [I>=2σR₁=0.0466, wR₂=0.0975 (I)]

Final R indexes [all data] R₁=0.0394, wR₂=0.1026

Largest diff. peak/hole/0.859/−0.508

e A⁻³

The sensitivity of the reagent was tested by hammer blow and with a drop-weight impact machine. The hammer test is an initial indication of the sensitivity of the molecule to an outside impact stimulus. Reagent in amount of 1 g were placed on a clean steel surface of a witness plate and hit with a hammer (250 g). No fume, heavy smoke, sparks, explosion, or heat were recorded, suggesting that this molecule is shock insensitive. For the fall-hammer test, MP-3 Falling Hammer equipment was used. Sample of the reagent (200 mg) was placed on a clean steel surface and a 1 kg hammer was raised to a predetermined height (0.5 m and 0.8 m) by a manual crank. The hammer was dropped from various height onto the striker. No effects have been recorded.

Although we encountered no incidents while synthesizing this molecule or products reported herein, safety precautions must be taken such as wearing safety glasses, protected shield, full body protective clothing, etc. Safety regulation of acyl nitrate is well documented in the following reference:Louw, R. e-EROS Encycl. Reagents Org. Synth. 2001, DOI: 10.1002/047084289X.ra032. For the use of multi kilograms of acetyl nitrate in the synthesis, see the following reference: Hoare, J.; Duddu, R.; Damavarapu, R. Org. Process Res. Dev. 2016, 20, 683-686).

### EXAMPLE 2

The procedure for synthesis of 6-nitro-N-nitrosaccharins:

In a 250 mL three necked round bottom flask equipped with dropping funnel, air outlet and stirring bar was placed N-saccharin (10.0 g, 36.63 mmol) in acetic anhydride (28.2 mL, 0.30 mol). The solution was cooled to 0-5° C. with ice-bath and concentrated nitric acid (28.2 mL, 0.67 mol) was added dropwise to the solution during 30 minutes, while dry air being bubbled through the solution rapidly in order to remove excess of nitrogen oxides. 6-nitrosaccharin was completely dissolved once all nitric acid was added. The reaction mixture was stirred at 5-10° C. during 4 hours with constant bubbling of air through the liquid. The reaction mixture was placed to freezer for hours to complete precipitation of the product. The precipitate was collected on a sintered glass filter, washed with cold chloroform and dried under high vacuum until dryness (9.6 g, 96% yield). The product is a light-yellow (almost white) powder/crystalline compound.

M.p./Decomposition temperature 174-176° C. (mass loss −50%, determined by thermogravimetric analysis);

¹H-NMR (500 MHz, CD3CN) : δ=9.07 (d, J=2.1 Hz, 1H), 8.76 (dd, J=8.5, 2.0 Hz, 1 H), 8.43 (d, J=8.4 Hz, 1H);

¹³C-NMR (125 MHz, CD₃CN): δ=118.3, 112.8, 128.5, 130.6, 135.4, 150.3, 152.9;

IR (ATR, neat):3073, 1732, 1601, 1529, 1424, 1347, 1180, 1064, 1024, 786, 737, 649, 490;

Analytically calculated for C₇H₃N₃O₇S: C 3.78, H 1.11, N 15.38 found: C 30.81, H 1.19, N 15.50.

Colorless crystals of compound Ib were obtained by slow evaporation from a saturated solution in chloroform/acetonitrile 1:1.

Crystal data and structure refinement of compound Ib:

Empirical formula C7H3N3O7S

Formula weight 273.18

Temperature/K 100.0

Crystal system orthorhombic

Space group IT number 61

Space group name P b c a

a/Å 9.4437(3)

b/Å 13.7774(5)

c/Å 14.6729(6)

α/° 90.0

β/° 90.0

γ/° 90.0

Volume/Å3 1909.09(12)

Z 8

ρcalcg/cm3 1.901

μ/mm−1 0.377

F(000) 1104.0

Crystal size/mm3 0.16×0.3×0.41

Radiation MoKα (λ=0.71073)

2θ range for data 2.174 to 29.565 collection/°

Index ranges −9≤h≤9, −11≤k≤11, −26≤1≤26

Reflections collected 22172

Independent reflections 2778

Data/restraints/parameters 2778/0/163

Goodness-of-fit on F2 1.174

Final R indexes [I>=2σR1=0.0586, wR2=(I)] 0.1093

Final R indexes [all data] R1=0.0436, wR2=0.1320

Largest diff. peak/hole/0.597/−0.576

e Å-3

### EXAMPLE 3

Representative general procedure I for nitration of arenes:

A 50 mL vessel was charged with compound Ia (1.3 equiv., 6.5 mmol) and sealed under nitrogen atmosphere. Arene (1.0 equiv., 5 mmol) and HFIP (10 mL) were added and the reaction mixture was heated at 55° C. for 2-19 hours, depending on the substrate. After cooling to room temperature, the solvent was removed in vacuum, and the product was purified by flash column chromatography (SiO₂, ethyl acetate/n-hexane gradient).

### EXAMPLE 4

Representative general procedure II for nitration of arenes:

A 50 mL vessel was charged with compound Ib (1.3 equiv., 6.5 mmol), Mg(ClO₄)₂ (0.5 mmol) and sealed under nitrogen atmosphere. Arene (1.0 equiv., 5 mmol) and CH₃CN (10 mL) were added and the reaction mixture was heated at 85° C. for 5-19 hours depending on the substrate. After cooling to room temperature, the solvent was removed in vacuum, and the product was purified by flash column chromatography (SiO2, ethyl acetate/n-hexane gradient).

### EXAMPLE 5—Comparative Experiment

The following procedure described by Kozlova et al. was investigated: An equimolar amount of nitronium tetrafluoroborate was added at −30° C. to a stirred suspension of 2 g of the imide salt of saccharin in 20 mL of the absolute acetonitrile, and the mixture was stirred at this temperature for 20-30 minutes. The precipitate was removed by filtration, and the filtrate was evaporated. The solid residue was washed with a mixture of hexane with methylene chloride. Despite multiple attempts, the proposed synthesis was never successful since no product formation could be observed at all.

1. N-nitrosaccharin of the general formula (I), 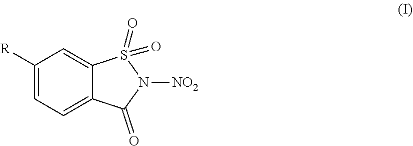
wherein R is either a hydrogen (H) or a nitro group (NO₂).

2. N-nitrosaccharin according to claim 1 of the formula (Ia)

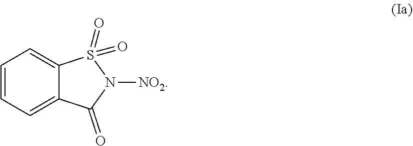 [SMILES: O=C1c2ccccc2S(=O)(=O)N1[N+](=O)[O-]]

3. 6-Nitro-N-nitrosaccharin according to claim 1 of the formula (Ib)

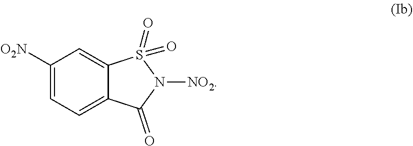 [SMILES: O=C1c2ccc([N+](=O)[O-])cc2S(=O)(=O)N1[N+](=O)[O-]]

4. N-nitrosaccharin according to claim 1, wherein the N-nitrosaccharin of general formula (I) is in crystalline form.

5. A method for the preparation of N-nitrosaccharin of the general formula (I) according to claim 1 comprising the following steps:
reacting N-saccharin of the general formula (II), 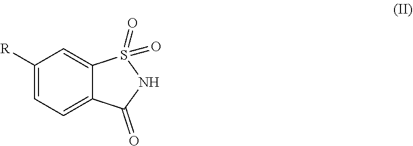
wherein R is either a hydrogen or a nitro group, in the presence of nitric acid, to obtain N-nitrosaccharin of general formula (I).

6. The method according to claim 5, wherein N-saccharin of the general formula (II) is dissolved in an organic anhydride.

7. The method according to claim 5, wherein the molar ratio of N-saccharin to concentrated nitric acid is between 500 to 1 and 10 to 1.

8. The method according to claim 5, wherein solution comprising N-saccharin of the general formula (II) dissolved in the organic solvent is cooled down below 15° C., during the addition of nitric acid is completed.

9. The method according to claim 5, wherein the reaction mixture is stirred for 1 to 24 hours.

10. The method according to claim 5, wherein N-nitrosaccharin of the general formula (I) is obtained with a yield of at least 50%.

11. The method according to claim 5, wherein the solvent is removed to obtain the N-nitrosaccharin of the general formula (I) in crystalline form.

12. A method for applying N-nitrosaccharin of the general formula (I), 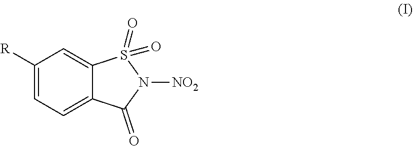
with R being either hydrogen (H) or a nitro group (NO₂),
as nitrating agent of a compound A in an electrophilic substitution,
wherein compound A comprises at least one substituted or unsubstituted aromatic or heteroaromatic ring.

13. The method according to claim 12, wherein the electrophilic substitution is an ipso-substitution.

14. The method for applying N-nitrosaccharin according to claim 12, wherein the aromatic or heteroaromatic ring of compound A comprises a leaving group, wherein the leaving group Y is selected from the group consisting of halogen atoms (I, Br, Cl, F), SO₃H, Si(CH₃)₃, tosyl, mesyl, nosyl, brosyl, tresyl, dansyl, trifyl, hydroxides, alkoxides, amides, acetyl substituents and tert-alkyl substituents.
